(19) United States
(12) Patent Application Publication
(10) Pub. No.: US 2025/0334581 A1
(43) Pub. Date: Oct. 30, 2025

(54) DOUBLY LABELED WATER WITH ENHANCED PROTOCOL

(71) Applicant: Calorify Inc., Scotts Valley, CA (US)

(72) Inventors: Hari Mix, Los Gatos, CA (US); Nolan Meyer, Scotts Valley, CA (US); Erica Svendahl, Los Gatos, CA (US)

(21) Appl. No.: 19/262,863

(22) Filed: Jul. 8, 2025

Related U.S. Application Data
(63) Continuation-in-part of application No. 18/408,172, filed on Jan. 9, 2024.

(51) Int. Cl. G01N 33/58

(52) U.S. Cl. CPC G01N 33/58; G01N 2458/15

(57) ABSTRACT

Doubly labeled water with enhanced protocol. One embodiment is a method including providing, to a user, a doubly labeled water (DLW) dose for the user to ingest, the DLW dose including deuterium and oxygen-18, wherein an amount of the deuterium is less than 0.12 grams per kilogram (g/kg) of body water of the user, and wherein an amount of the oxygen-18 is less than 0.18 g/kg of body water of the user. The method also includes receiving, from the user, a non-cooled shipment of urine samples collected in connection with ingestion of the DLW dose, wherein the urine samples remain uncooled after collection and during transit for a period of up to 24 days. The method further includes processing the urine samples with a liquid water isotope analyzer to determine one or more metabolic parameters of the user.

### REFERENCE TO RELATED APPLICATIONS

The present application is a Continuation-in-Part of U.S. patent application Ser. No. 18/408,172, filed Jan. 9, 2024 and titled “Doubly Labeled Water with Enhanced Protocol,” which is incorporated hereby in its entirety by reference.

### BACKGROUND

### Field

The present disclosure relates to doubly labeled water (DLW), and in particular, to techniques for improving the process of DLW sample analysis.

### Background

Doubly labeled water (DLW) is a technique used to measure energy expenditure and metabolic processes of a person over time. It involves using water molecules that are “doubly labeled” with stable isotopes, typically deuterium and oxygen-18. After a person drinks water enriched with these isotopes, their urines samples are collected at specific time intervals. An isotope analyzer processes the urine samples to determine the rate at which the person's body eliminated deuterium and oxygen-18. This rate of elimination of the isotopes is used to calculate the person's total energy expenditure (TEE) or other metabolic parameters.

DLW analysis is often used in the context of academic or research laboratories conducting studies in various scientific fields. These studies typically require close supervision and controlled conditions during the administration of the DLW and subsequent collection of biological samples. Existing techniques for performing DLW analysis have remained stagnant due, at least in part, to an emphasis on controlling variables for consistent study design. Comparatively few laboratories can provide DLW analysis as a service for individuals remotely located from the laboratory, and there is a need for updated processes that address the cost and turnaround time of providing DLW analysis results to individuals.

### SUMMARY

According to an aspect, a method is disclosed where the method includes providing, to a user, a doubly labeled water (DLW) dose for the user to ingest, the DLW dose including deuterium and oxygen-18, wherein an amount of the deuterium is less than 0.12 grams per kilogram (g/kg) of body water of the user, and wherein an amount of the oxygen-18 is less than 0.18 g/kg of body water of the user. The method also includes receiving, from the user, a non-cooled shipment of urine samples collected in connection with ingestion of the DLW dose, wherein the urine samples remain uncooled after collection and during transit for a period of up to 24 days. The method further includes processing the urine samples with a liquid water isotope analyzer to determine one or more metabolic parameters of the user.

### BRIEF DESCRIPTION OF THE DRAWINGS

FIG. 1 is a block diagram illustrating a process of performing doubly labeled water (DLW) analysis in an example embodiment.

FIG. 2 is a flowchart illustrating a method of performing DLW analysis in an example embodiment.

FIG. 3 is a flowchart illustrating another method of performing DLW analysis in an example embodiment.

FIG. 4 is a graph showing experimental results of deuterium measurements over time for different temperatures.

FIG. 5 is a graph showing experimental results of oxygen-18 measurements over time for different temperatures.

FIG. 6 is a graph showing dosing amounts determined from experimental results and compared to the prior art.

FIG. 7 is a bar graph illustrating a cost comparison by dosing formula of embodiments herein as compared to the prior art.

FIG. 8 is a graph showing experimental results of total energy expenditure standard deviation per dosages of oxygen-18 in illustrative embodiments.

FIG. 9 is a graph showing experimental results of total energy expenditure standard deviation per dosages of deuterium in illustrative embodiments.

### DETAILED DESCRIPTION

The following modes, features or aspects, given by way of example only, are described in order to provide a more precise understanding of the subject matter of several embodiments.

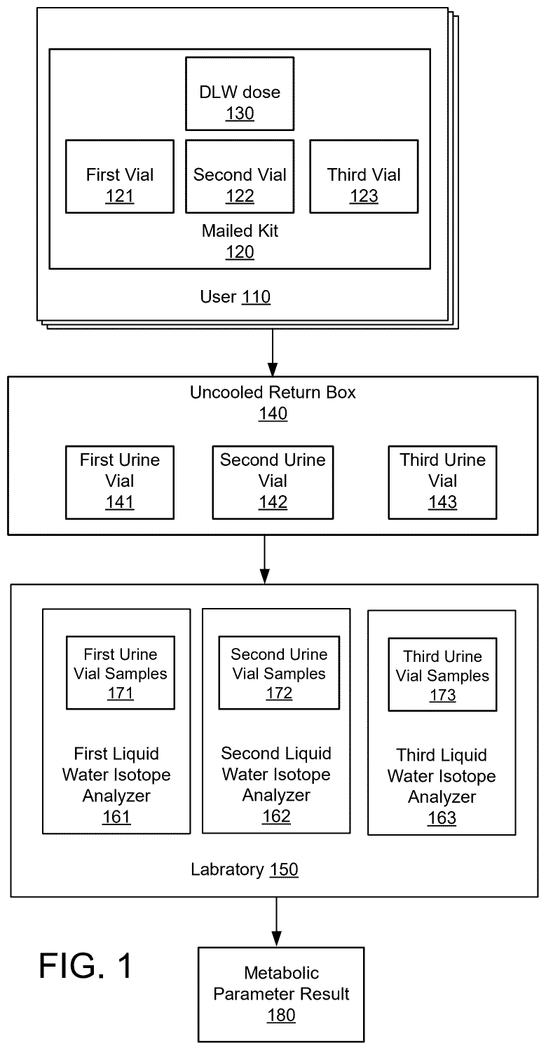

FIG. 1 is a block diagram illustrating a process of performing doubly labeled water (DLW) analysis in an example embodiment. In general, a user 110 or person may be provided with a mailed kit 120 including a DLW dose 130 enriched with isotopes, and vials 121-123, which are initially empty. Before and/or after drinking DLW dose 130, user 110 collects urine in vials 121-123 at specific time intervals, producing urine vials 141-143. Urine vials 141-143 are stored and transported in an uncooled return box 140 and provided to laboratory 150. Laboratory 150 includes one or more liquid water isotope analyzers 161-163 configured to measure an isotopic composition of urine sampled from urine vials 141-143 (i.e., urine vial samples 171-173). After processing urine samples, laboratory 150 may calculate and provide a metabolic parameter result 180 for user 110. Examples of metabolic parameter result 180 include total energy expenditure (TEE), body composition, hydration, and training level.

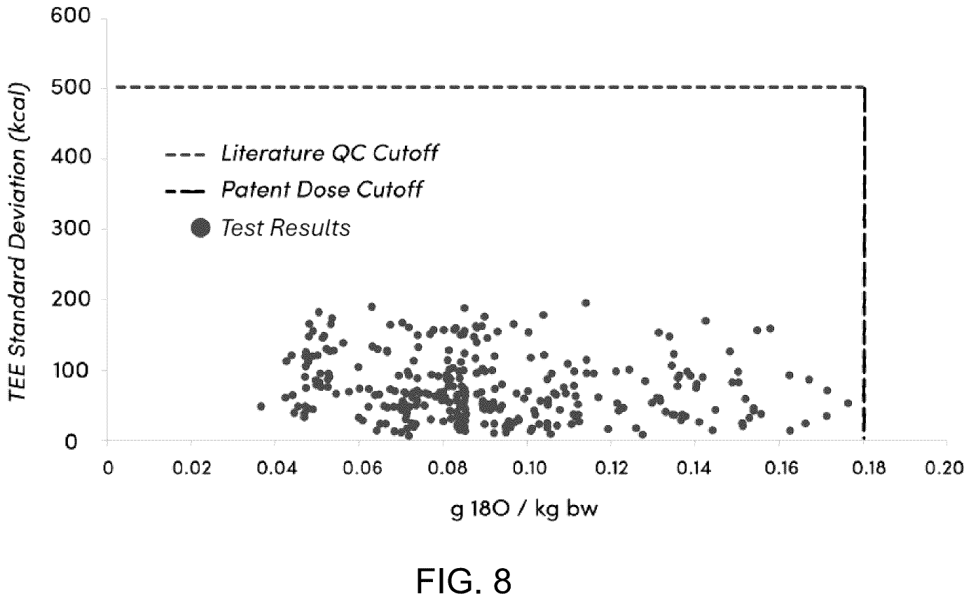

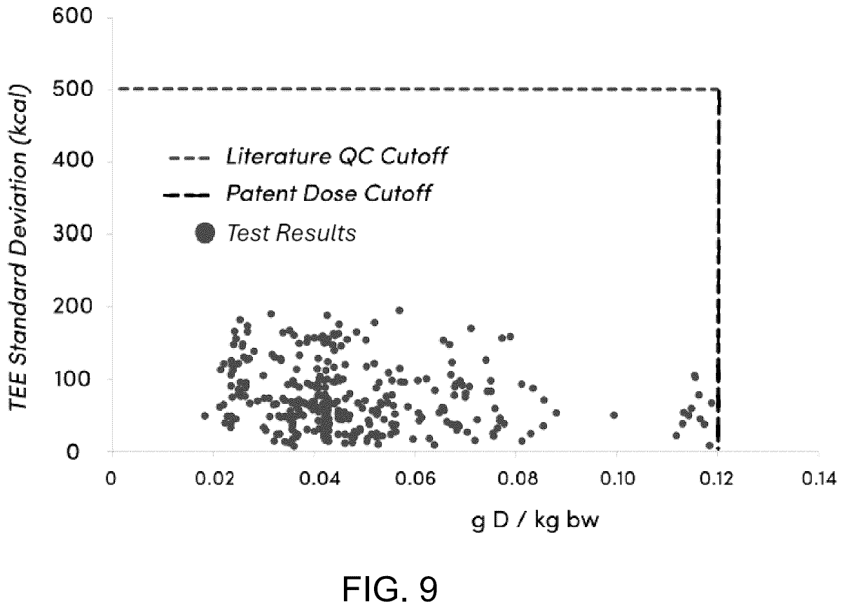

As will be described in greater detail below, the process of performing DLW analysis is enhanced by a combination of one or more of the following three components. First, through experimentation, the inventors of this Application have determined that DLW dose 130 provided to user 110 can be created with a reduced amount of isotope(s) compared to conventional dosages without compromising the accuracy of metabolic parameter result 180, resulting in significant cost savings that can be passed on to user 110. The reduced amount of isotope(s) compared to conventional dosages is further discussed in FIGS. 8 and 9, together with their associated text. Second, experimentation also showed that urine vials 141-143 collected by user 110 can be stored and transported in an uncooled state (e.g., uncooled return box 140) without compromising the accuracy of metabolic parameter result 180, a significant departure from currently accepted practices. This discovery also significantly reduces costs for user 110 as it enables mailed kit 120 and/or uncooled return box 140 to comprise smaller and lighter packages in the absence of cooling items (e.g., foam insulation and ice, dry ice, or gel packs) considered mandatory in conventional DLW analysis. Third, laboratory 150 is enhanced with one or more parallel measurement techniques to improve the efficiency and/or accuracy of metabolic parameter result 180 returned to user 110.

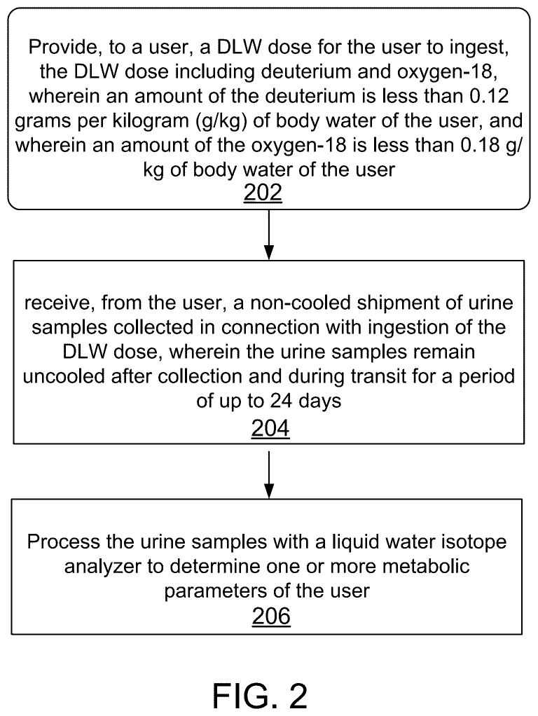

FIG. 2 is a flowchart illustrating a method of performing DLW analysis in an example embodiment. The steps of the flowcharts herein are described with reference to FIG. 1, but those skilled in the art will appreciate that the method may be performed for multiple different users and in other scenarios, environments, and/or systems. The steps of the flowcharts described herein are not all inclusive and may include other steps not shown.

In step 202, DLW dose 130 is provided to user 110, wherein DLW dose 130 includes deuterium and oxygen-18, wherein an amount of the deuterium is less than 0.12 grams per kilogram (g/kg) of body water of user 110, and wherein an amount of the oxygen-18 is less than 0.18 g/kg of body water of user 110. In one embodiment, DLW dose 130 includes less than 0.08 g/kg of deuterium and less than 0.12 g/kg of oxygen-18. In another embodiment, DLW dose 130 includes approximately 0.035 g/kg body water of deuterium-labeled water and approximately 0.070 g/kg body water of oxygen-18 labeled water. The determination of amounts of deuterium and oxygen-18 in DLW dose 130 and advantages thereof is discussed in greater detail below.

In some embodiments, DLW dose 130 is provided in a watertight container as an item of mailed kit 120 (e.g., packaged and transported via postal services for delivery to a specified recipient or destination, such as a home address of user 110). In one embodiment, DLW dose 130 comprises approximately one fluid ounce of liquid in a container capable of storing approximately four fluid ounces of liquid. In addition to DLW dose 130, mailed kit 120 may include other items such as vials 121-123, uncooled return box 140, return/destination postage and/or address information, one or more cups for collecting urine, one or more pipettes or similar devices for transferring urine from a cup to a vial, and/or user instructions for collecting urine samples at specific time intervals after drinking DLW dose 130. Alternatively, in some embodiments, DLW dose 130 may be provided to user 110 independently or separate from mailed kit 120. Mailed kit 120 and/or DLW dose 130 may be provided by an entity that operates, or partners with, laboratory 150 configured to process urine samples to determine isotope composition.

In step 204, laboratory 150 receives, from user 110, a non-cooled shipment of urine samples collected in connection with ingestion of DLW dose 130, wherein the urine samples remain uncooled after collection and during transit for a period of up to twenty-four days. That is, laboratory 150 may receive uncooled return box 140 storing urine vials 141-143 produced by user 110 in accordance with urine collection instructions and delivered via postal services. As described in greater detail below, inventors of this Application have discovered through experimentation that urine vials 141-143, having never been cooled, may be processed with sufficient accuracy even after accounting for adequate time for a customer's testing period (e.g., up to nine days) and a worse-case shipping scenario (e.g., up to fifteen days in transit) for up to a total period of twenty-four days.

In step 206, laboratory 150 processes the urine samples with one or more liquid water isotope analyzers 161-163 to determine one or more metabolic parameters of user 110. Metabolic parameters may comprise at least one numerical value or range for one or more metabolic categories such as energy balance (e.g., TEE), body composition, hydration, and/or training level. Measurements and/or calculated values may be included in metabolic parameter result 180 and transmitted to user 110 in any suitable form, such as e-mail, login portal, mobile application, text, and/or postal services. In one embodiment, laboratory 150 includes multiple liquid water isotope analyzers 161-163 to process multiple urine vial samples 171-173 of user 110 in parallel. For instance, using a syringe or similar device, one or more first urine vial samples 171 may be taken from first urine vial 141 of user 110 and placed in first liquid water isotope analyzer 161 for measuring, one or more second urine vial samples 172 may be taken from second urine vial 142 of user 110 and placed in second liquid water isotope analyzer 162 for measuring, and one or more third urine vial samples 173 may be taken from third urine vial 143 of user 110 and placed in third liquid water isotope analyzer 163 for measuring. Additional details related to parallel measurement techniques are discussed below.

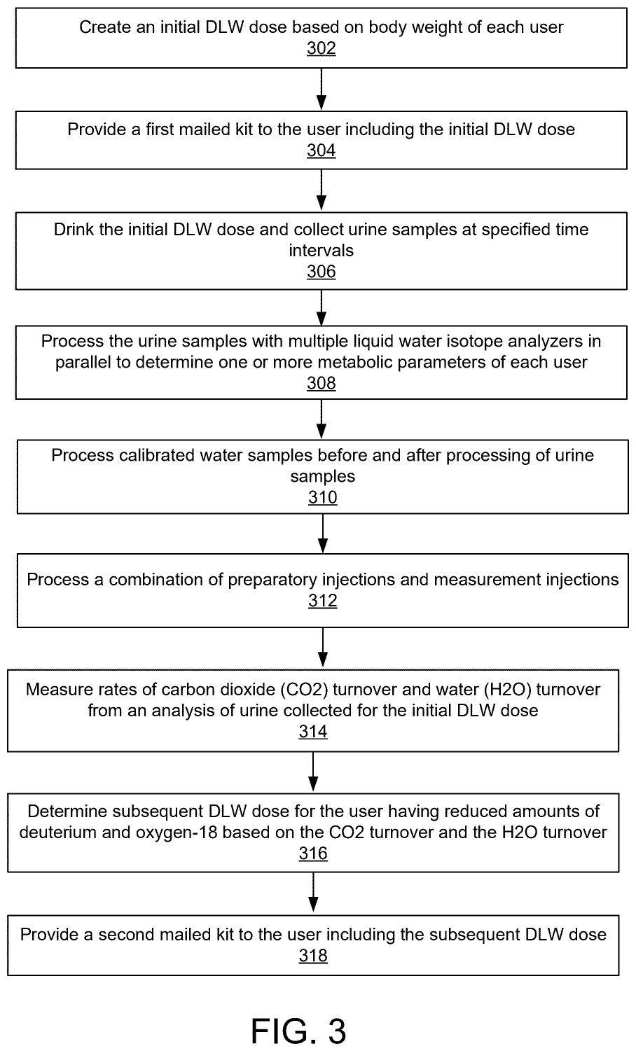

FIG. 3 is a flowchart illustrating another method of performing DLW analysis in an example embodiment. The steps of FIG. 3 may comprise additional, alternative, and/or optional steps to those discussed above with respect to FIG. 2. The steps of the flowcharts described herein are not all inclusive, may include other steps not shown, and may be performed in an alternative order.

In step 302, an initial DLW dose is created based on a body weight of each user 110. For instance, an amount of deuterium and/or oxygen-18 provided in the DLW dose may be customized for user 110 based on a combination of a body weight of user 110 and whether user 110 is an athlete. In any case, the initial DLW dose includes a sufficient amount of deuterium and/or oxygen-18 to spike levels of user 110 and remain above baseline at a time of collecting the last urine sample (e.g., approximately one week after ingestion of DLW dose).

In step 304, a first mailed kit is provided to each user 110, the first mailed kit including the initial DLW dose. In step 306, each user 110 drinks the initial DLW dose and collects urine samples at instructed time intervals. In one embodiment, user 110 is directed to collect three samples or vials, including first urine vial 141 for storing urine collected before ingestion of the DLW dose (e.g., to ascertain a background or baseline level of isotope composition), second urine vial 142 for storing urine collected approximately four to six hours after ingestion of the DLW dose, and third urine vial 143 for storing urine collected approximately one week after ingestion of the DLW dose. User 110 may record a date and time of collecting each vial and drinking DLW dose and include the recordation in uncooled return box 140 or otherwise provide the information to laboratory 150.

In one embodiment, urine vials 141-143, which are initially vials 121-123 before collection, comprise receptacles suitable for transporting liquid and are sized to collectively fit within uncooled return box 140 having dimensions approximately 4.5 inches (11.5 cm) in width, 3.0 inches (7.6 cm) in height, and 1.0 inches (2.5 cm) in depth. Advantageously, the relatively small package size and absence of cooling pack in uncooled return box 140 reduces shipping costs significantly. That is, the absence of cooling items in mailed kit 120 and/or uncooled return box 140 results in significant cost savings not obtainable under previously accepted DLW analysis standards.

In step 308, laboratory 150 processes the urine samples with multiple liquid water isotope analyzers 161-163 in parallel to determine one or more metabolic parameters of each user 110. For instance, first liquid water isotope analyzer 161 may measure samples taken from first urine vials 141 of a plurality of users 110 (e.g., samples of users 110 which were collected prior to ingestion of the DLW dose). Second liquid water isotope analyzer 162 may measure samples taken from second urine vials 142 of a plurality of users 110 (e.g., samples of users 110 which were collected approximately four to six hours after ingestion of the DLW dose). And third liquid water isotope analyzer 163 may measure samples taken from third urine vials 143 of a plurality of users 110 (e.g., samples of users 110 which were collected approximately one week after ingestion of the DLW dose). The use of multiple liquid water isotope analyzers 161-163 to measure samples in parallel provides a technical benefit in terms of improved throughput at laboratory 150 and faster turnaround of results for users 110. In addition, the parallel configuration allows two additional techniques to be implemented not used in conventional DLW processes, as further described in connection with steps 310-312.

In step 310, laboratory 150 processes one or more calibrated water samples before and after processing one or more urine samples of user 110. A calibrated water sample has a known amount of deuterium and/or oxygen-18 and is used to calibrate liquid water isotope analyzers 161-163 and improve the accuracy of unknown urine samples. For instance, a run pattern for measuring the isotope composition of urine from one or more first vials (e.g., collected before DLW ingestion) may comprise: (1) process a first calibrated sample having a first known composition, (2) process a second calibrated sample having a second known composition, (3) process a third calibrated sample having a third known composition, (4) process a first urine sample from a first vial of a first user, (5) process a second urine sample from the first vial of the first user, (6) process a first urine sample from a first vial a second user, (7) process a second urine sample from the first vial of the second user, (8) process the first calibrated sample again, (9) process the second calibrated sample again, and (10) process the third calibrated sample again.

Implementing this run pattern, the analyzer may obtain an accurate isotope composition of the first user's first vial from steps (4)-(5). If the first measurement and subsequent duplicate measurement (e.g., measurement result from steps (4)-(5)) are not sufficiently close to one another, additional sample measurements may be taken for that user until the values sufficiently converge. This example run pattern includes sample measurements taken for a second user in steps (6)-(7), though it will be appreciated that multiple additional users may be added to the run for efficiency. A similar run may be performed for each user's other two vial samples on two other analyzers, and this data may be input into a computer to calculate each user's metabolic parameters.

Here, vials which are similar in composition among different users (e.g., third vials collected approximately one week after ingestion of DLW) are grouped and run through the same analyzer. This parallel technique allows the calibrated water samples to have an isotope composition that is similar to the composition of the urine samples, resulting in highly accurate measurements. By contrast, prior techniques may run all three of a user's samples back-to-back through the same analyzer, and as a result, be forced to use calibrated water samples having composition values farther apart from the unknown samples, thus having reduced accuracy in comparison.

In one embodiment, each liquid water isotope analyzer 161-163 measures three calibrated water samples before and after measuring one or more user samples grouped by collection order/time, and the isotopic amount of the three calibrated water samples is specific to each liquid water isotope analyzer 161-163 to be similar to, or correspond with, an isotopic composition of that group of user samples for enhanced accuracy. Suppose, for example, that first liquid water isotope analyzer 161 is assigned to measure first urine vials 141 of a plurality of users 110 collected prior to ingestion of the DLW dose, second liquid water isotope analyzer 162 is assigned to measure second urine vials 142 of a plurality of users 110 collected approximately four to six hours after ingestion of the DLW dose, and third liquid water isotope analyzer 163 is assigned to measure third urine vials 143 collected approximately one week after ingestion of the DLW dose. Through experimentation, the inventors of this Application have determined a range of isotopic values of the three calibrated water samples for each liquid water isotope analyzer 161-163 which contains the isotopic composition of unknown urine samples for that group while being proximate in value for improved measurement accuracy.

In particular, in a further embodiment, first liquid water isotope analyzer 161 measures a first group of three calibrated standard water samples having an isotopic composition which contains the values of first urine vials 141, wherein the isotopic composition spans less than 153.2 per mil in deuterium and less than 18.5 per mil in oxygen-18. Second liquid water isotope analyzer 162 measures a second group of three calibrated standard water samples having an isotopic composition which contains the values of second urine vials 142, wherein the isotopic composition spans less than 274.8 per mil in deuterium and less than 36.4 per mil in oxygen-18. Third liquid water isotope analyzer 163 measures a third group of three calibrated standard water samples having an isotopic composition which contains the values of third urine vials 143, wherein the isotopic composition spans less than 191.0 per mil in deuterium and less than 26.3 per mil in oxygen-18.

In step 312, laboratory 150 processes a combination of preparatory injections and measurement injections. Step 312 may be performed in conjunction with step 310 described above. For instance, each sample processing step (1)-(10) described above in connection with step 310 may comprise a number of preparatory injections and measurement injections. Preparatory injections are samples that run or process through the isotope analyzer without any measurement taken, whereas measurement injections are measured by the analyzer and thus take slightly more time to process. For instance, a preparatory injection may take approximately sixty seconds to process through the analyzer and a measurement injection may take approximately ninety seconds to process through the analyzer. The preparatory injections serve to reduce the “memory effect” an isotope analyzer may have so that the measurement injections are more accurate. An injection in this context may refer to a laboratory worker or automated process using a syringe or similar device to inject a small liquid drop of a sample or vial into the isotope analyzer.

Here, the parallel measurements and reduced range of isotope composition values seen by each isotope analyzer enables laboratory 150 to reduce the number of preparatory injections. That is, the memory effect, or residual influence of previously analyzed samples on subsequent measurements, is substantially reduced because of the reduced isotope range between multiple urine samples measured by the same isotope analyzer. This allows the number of preparatory injections to reduce from approximately twelve preparatory injections typically used to less than six preparatory injections for each measurement of the sample. For instance, processing or measuring a sample may include less than six preparatory injections followed by less than eight measurement injections. A technical benefit is thus provided by eliminating many preparatory injections across each processing run, resulting in significantly improved throughput for laboratory 150. In one embodiment, liquid water isotope analyzers 161-163 comprise off-axis integrated cavity output spectrometer (OA-ICOS) analyzers. In other embodiments, liquid water isotope analyzers 161-163 may comprise other types of machines configured to analyze isotope amounts using laser absorption spectrometry or mass spectrometry.

In step 314, laboratory 150 measures rates of carbon dioxide (CO2) turnover and water (H2O) turnover from an analysis of urine collected for the initial DLW dose. In step 316, laboratory 150 determines a subsequent DLW dose for a user based on the measured CO2 and H2O turnover of the user, wherein the subsequent DLW dose has reduced amounts of deuterium and oxygen-18 relative to the initial DLW dose for the user. That is, the initial DLW dose may include isotope amounts that are estimated but overly sufficient to ensure a margin of safety for obtaining accurate test results. After processing the first kit samples, the measured CO2 and H2O turnover can be used to dose the customer more accurately, further reducing DLW dose costs.

In step 318, laboratory 150 provides a second mailed kit to the user including the subsequent DLW dose. As an example, suppose laboratory 150 measures the turnover of a user as 30% less than originally dosed for in the initial DLW dose, laboratory 150 may then create DLW doses for future orders of that user to have approximately 20% less dose amounts relative to the initial dose such that there is approximately a 10% buffer of sufficient dose amounts to still obtain accurate measurements.

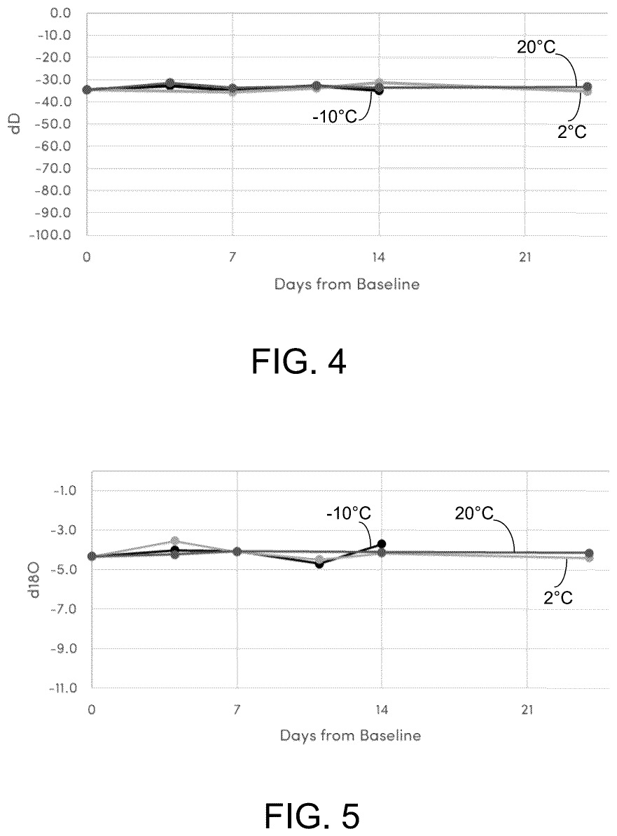

FIG. 4 is a graph showing experimental results of deuterium measurements over time for different temperatures. FIG. 5 is a graph showing experimental results of oxygen-18 measurements over time for different temperatures. The inventors of this Application performed a shelf-life test with urine samples to determine the necessity of frozen storage and/or shipments. The vertical axis of the graphs indicates the deviation of the sample's isotopic composition compared to a baseline standard. That is, isotopic composition was measured and compared to baseline after 4, 7, 11, 14, and 24 days when stored in a freezer (−10° C.), a refrigerator (2° C.), and at room temperature (20° C.). A maximum window of 24 days was selected to allow adequate time for a customer's testing period (up to 9 days) and a worst-case shipping scenario (up to 15 days in transit). Room temperature deuterium and oxygen-18 deviated just 1.3 per mil (‰) and 0.3‰, respectively, indicating virtually no change in isotopic composition despite the significant departure from standard DLW practices.

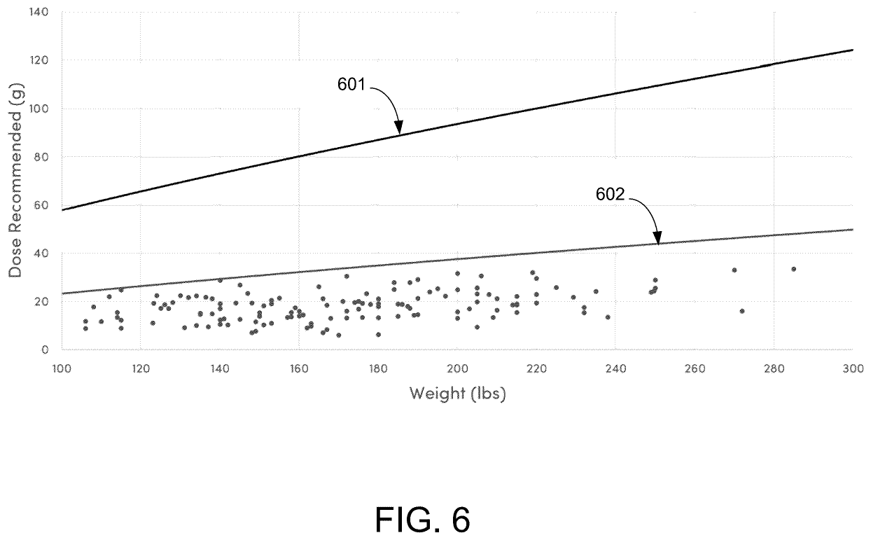

FIG. 6 is a graph showing dosing amounts determined from experimental results and compared to the prior art. Line 601 represents a dosing curve as described by scientific literature, and line 602 represents a dosing curve as described by embodiments herein. In particular, line 601 shows subjects dosed with more than 0.12 g/kg body water of 99.8% deuterium-labeled water and 1.8 g/kg body water of 10.0% oxygen-18-labeled water, as described in the non-patent literature cited herewith. By contrast, line 602 is based on plotted dots representing a recommended dose amount of over one hundred and thirty customers determined by experiment. Each recommended dose amount was calculated based on a customer's measured carbon dioxide (CO2) turnover and water (H2O) turnover. By determining the rate at which a customer's dose left their body, their actual dosing need was calculated. Line 602, or dosing curve, was fit to the plotted measurement results and indicates a significantly reduced dosing amount of approximately 0.035 g/kg body water of deuterium and approximately 0.070 g/kg body water of oxygen-18.

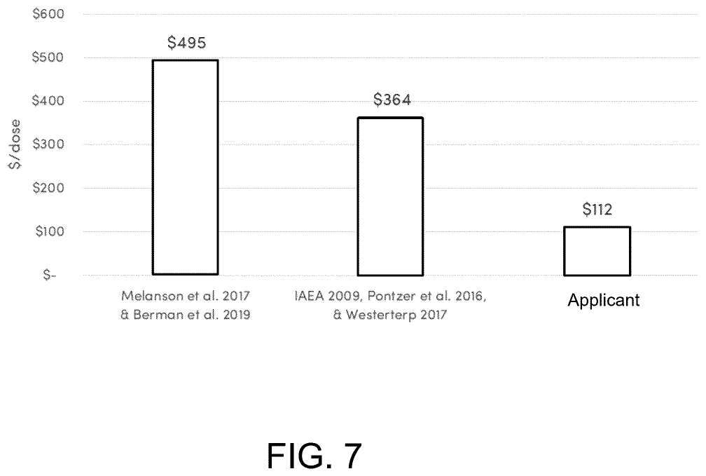

FIG. 7 is a bar graph illustrating a cost comparison by dosing formula of embodiments herein as compared to the prior art. Assuming an average user weight of 180 pounds at 73% average fat-free mass, Applicant's DLW dosing amount reduces costs by approximately $252, or 69% reduction in cost, per test. That is, the experiments demonstrated that the average dose of DLW can be substantially reduced such that a 69% reduction in the amount of deuterium and/or oxygen-18 (as compared to non-patent literature cited herewith) still allowed for an enrichment of at least 8‰ deviation in final oxygen-18 values. Accordingly, the experimentation demonstrated that lowering the DLW dosage amount had a disproportionate and less-than-expected influence on measurement accuracy, thus showing an ability to provide users with accurate metabolic results at significantly reduced cost.

FIG. 8 is a graph showing experimental results of total energy expenditure (TEE) standard deviation per dosages of oxygen-18 in illustrative embodiments. The inventors of this application performed reduced dosing of oxygen-18 (18O) to determine any relationship between oxygen-18 dose size and standard deviation in total energy expenditure. The vertical axis of the graph indicates the total energy expenditure (TEE) standard deviation in kilocalories (kcal), and the horizontal axis of the graph indicates the dose of oxygen-18 (e.g., grams (g) of oxygen-18 (18O) per kilogram (kg) of body water (bw)). The test results are plotted on the graph and the literature quality control (QC) cutoff is shown as a short-dashed line (e.g., at about 500 kcal) along with the patent dose cutoff of 0.18 grams oxygen-18 per kilogram body water as a long-dashed line (e.g. at about 0.18 g 18O/kg bw). To obtain the test results, each user was given a dose of oxygen-18 less than 0.18 g/kg of body water of the user (as described in various embodiments disclosed herein, including step 202 of FIG. 2). The test results shown in the embodiments of FIG. 8 show an R² value of 0.0232 (calculated from least squares regression lines through the data points for the test results shown in FIG. 8), which demonstrates that there is not a relationship between the oxygen-18 dose size and standard deviation in total energy expenditure.

FIG. 9 is a graph showing experimental results of total energy expenditure (TEE) standard deviation per dosages of deuterium in illustrative embodiments. The inventors of this application performed reduced dosing of deuterium (D) to determine any relationship between deuterium dose size and standard deviation in total energy expenditure. The vertical axis of the graph indicates the total energy expenditure (TEE) standard deviation in kilocalories (kcal), and the horizontal axis of the graph indicates the dose of deuterium (e.g., grams (g) of deuterium (D) per kilogram (kg) of body water (bw)). The test results are plotted on the graph and the literature quality control (QC) cutoff is shown as a short-dashed line (e.g., at about 500 kcal) along with the patent dose cutoff of 0.12 grams deuterium per kilogram body water as a long-dashed line (e.g. at about 0.12 g D/kg bw). To obtain the test results, each user was given a dose of deuterium less than 0.12 g/kg of body water of the user (as described in various embodiments disclosed herein, including step 202 of FIG. 2). The test results shown in the embodiments of FIG. 9 show an R² value of 0.0285 (calculated from least squares regression lines through the data points for the test results shown in FIG. 9), which demonstrates that there is not a relationship between the oxygen-18 dose size and standard deviation in total energy expenditure.

The results shown in FIGS. 8 and 9 are advantageous because, for example, it is shown that the dose of oxygen-18 and/or deuterium that is necessary to make total energy expenditure measurements according to various embodiments disclosed herein (e.g., using the DLW methods) is less than previously known, and the lesser doses advantageously maintain a high level of accuracy. For example, existing literature described accepted standard deviations of up to 500 kcal (different analytical methods for measuring total energy expenditure were explored and it was found that across different instrumentation and compared to whole room calorimetry, there was no difference in error and the error was between 1.56 megajoule (MJ) and 1.70 MJ, or 372-406 kcal) or a wider accepted error on the order of 506 kcal (in one explicit quality control paper regarding cutoffs, which outlined cutoffs in regard to errors that came off of the instruments expressed in delta values). Advantageously, FIGS. 8 and 9 show that the test results fall well within these quality control cutoffs of 500 kcal and 506 kcal, demonstrating that there is no decrease in accuracy with the use of a reduced dose of DLW (as disclosed in the various embodiments described herein), and the disclosed embodiments greatly exceed the stated tolerances in the existing literature. In advantageous aspects, FIGS. 8 and 9 show that there is no correlation between Applicant's DLW dose amount and accuracy using the methods disclosed herein. In further advantageous aspects, Applicant's DLW dosing maintains a high level of accuracy, as well as reducing costs while maintaining the high level of measurement accuracy, thus showing an ability to provide users with accurate metabolic results at significantly reduced cost.

What is claimed is:

1. A method of performing Doubly Labeled Water (DLW) analysis, the method comprising:
providing, to a user, a DLW dose for the user to ingest, the DLW dose including an amount of deuterium less than 0.12 grams per kilogram (g/kg) of body water of the user, and an amount of oxygen-18 less than 0.18 g/kg of body water of the user;
receiving, from the user, a shipment of urine samples collected in connection with ingestion of the DLW dose; and
processing the urine samples with a liquid water isotope analyzer to determine one or more metabolic parameters of the user.

2. The method of claim 1, wherein:
the DLW dose includes approximately 0.035 g/kg body water of deuterium and approximately 0.070 g/kg body water of oxygen-18.

3. The method of claim 1, wherein:
the amount of the deuterium and the amount of the oxygen-18 in the DLW dose are each less than a typical DLW dose; and
there is no decrease in an accuracy of the determined one or more metabolic parameters of the user when the DLW dose is used instead of the typical DLW dose.

4. The method of claim 1, further comprising:
providing a first mailed kit to the user, the first mailed kit including an initial DLW dose of less than 0.12 g/kg of body water of the deuterium and less than 0.18 g/kg of body water of the oxygen-18;
measuring rates of carbon dioxide (CO2) turnover and water (H2O) turnover from an analysis of urine collected for the initial DLW dose;
determining a subsequent DLW dose for the user having reduced amounts of the deuterium and the oxygen-18 based on the CO2 turnover and the H2O turnover; and
providing a second mailed kit to the user including the subsequent DLW dose;
wherein the first mailed kit includes three vials including a first vial for storing urine collected before ingestion of the DLW dose, a second vial for storing urine collected approximately four to six hours after ingestion of the DLW dose, and a third vial for storing urine collected approximately one week after ingestion of the DLW dose.

5. The method of claim 1, wherein:
there is no correlation between the DLW dose and an accuracy of the one or more metabolic parameters.

6. The method of claim 4, further comprising:
providing, to each of a plurality of users, the first mailed kit including the DLW dose and the three vials;
receiving the three vials from each of the plurality of users;
processing urine samples from a plurality of first vials of the plurality of users in a first liquid water isotope analyzer;
processing urine samples from a plurality of second vials of the plurality of users in a second liquid water isotope analyzer; and
processing urine samples from a plurality of third vials of the plurality of users in a third liquid water isotope analyzer;
wherein processing urine samples comprises:
processing a first group of three calibrated standard water samples before and after processing one or more first vials of urine samples, wherein an isotopic composition of the first group of standard water samples spans less than 153.2 per mil in deuterium and less than 18.5 per mil in oxygen-18;
processing a second group of three calibrated standard water samples before and after processing one or more second vials of urine samples, wherein an isotopic composition of the second group of standard water samples spans less than 274.8 per mil in deuterium and less than 36.4 per mil in oxygen-18; and
processing a third group of three calibrated standard water samples before and after processing one or more third vials of urine samples, wherein an isotopic composition of the second group of standard water samples spans less than 191.0 per mil in deuterium and less than 26.3 per mil in oxygen-18; and
wherein each processing of a sample includes less than six preparatory injections and less than eight measurement injections.

7. The method of claim 4, further comprising:
providing, to each of a plurality of users, the first mailed kit including the DLW dose and the three vials;
receiving the three vials from each of the plurality of users;
processing urine samples from a plurality of first vials of the plurality of users in a first liquid water isotope analyzer;
processing urine samples from a plurality of second vials of the plurality of users in a second liquid water isotope analyzer; and
processing urine samples from a plurality of third vials of the plurality of users in a third liquid water isotope analyzer;
wherein processing urine samples comprises:
processing a first group of three calibrated standard water samples before and after processing one or more first vials of urine samples, wherein an isotopic composition of the first group of standard water samples spans less than 153.2 per mil in deuterium and less than 18.5 per mil in oxygen-18;
processing a second group of three calibrated standard water samples before and after processing one or more second vials of urine samples, wherein an isotopic composition of the second group of standard water samples spans less than 274.8 per mil in deuterium and less than 36.4 per mil in oxygen-18; and
processing a third group of three calibrated standard water samples before and after processing one or more third vials of urine samples, wherein an isotopic composition of the second group of standard water samples spans less than 191.0 per mil in deuterium and less than 26.3 per mil in oxygen-18; and
wherein processing the urine samples with multiple liquid water isotope analyzers in parallel to determine at least one metabolic parameter of each user.

8. The method of claim 1, wherein:
test results for a graph showing experimental results of total energy expenditure standard deviation in kilocalories per a plurality of dosages of oxygen-18 less than the DLW dose of 0.18 grams per kilogram of the body water of the user show an R-squared value of about 0.0232.

9. The method of claim 1,
test results for a graph showing experimental results of total energy expenditure standard deviation in kilocalories per a plurality of dosages of deuterium less than the DLW dose of 0.12 grams per kilogram of the body water of the user show an R-squared value of about 0.0285.

10. The method of claim 1, further comprising:
receiving body weight information of each user of a plurality of users; and
providing, to each user, a customized amount of deuterium and oxygen-18 in the DLW dose based on the body weight information.

11. A method of performing Doubly Labeled Water (DLW) analysis, the method comprising:
providing, to each of a plurality of users, a DLW dose for the user to ingest, the DLW dose including an amount of deuterium less than 0.12 grams per kilogram (g/kg) of body water of the user, and an amount of oxygen-18 less than 0.18 g/kg of body water of the user;
receiving, from each user, three vials of urine collected in connection with ingestion of the DLW dose; and
measuring samples of the three vials with one or more liquid water isotope analyzers to determine one or more metabolic parameters of each user.

12. The method of claim 11, wherein:
the DLW dose includes less than 0.08 g/kg of deuterium and less than 0.12 g/kg of oxygen-18.

13. The method of claim 11, wherein:
the DLW dose includes approximately 0.035 g/kg body water of deuterium and approximately 0.070 g/kg body water of oxygen-18.

14. The method of claim 11, further comprising:
measuring rates of carbon dioxide (CO2) turnover and water (H2O) turnover from an analysis of urine collected for the initial DLW dose;
determining a subsequent DLW dose for the user having reduced amounts of the deuterium and the oxygen-18 based on the CO2 turnover and the H2O turnover; and
providing the subsequent DLW dose to the user.

15. The method of claim 11, wherein:
the three vials include a first vial for storing urine collected before ingestion of the DLW dose, a second vial for storing urine collected approximately four to six hours after ingestion of the DLW dose, and a third vial for storing urine collected approximately one week after ingestion of the DLW dose; and
further comprising:
measuring, with a first liquid water isotope analyzer, urine samples from a plurality of first vials of the plurality of users;
measuring, with a second liquid water isotope analyzer, urine samples from a plurality of second vials of the plurality of users;
measuring, with a third liquid water isotope analyzer, urine samples from a plurality of third vials of the plurality of users;
measuring a first group of three calibrated standard water samples before and after measuring one or more first vials of urine samples, wherein an isotopic composition of the first group of standard water samples spans less than 153.2 per mil in deuterium and less than 18.5 per mil in oxygen-18;
measuring a second group of three calibrated standard water samples before and after measuring one or more second vials of urine samples, wherein an isotopic composition of the second group of standard water samples spans less than 274.8 per mil in deuterium and less than 36.4 per mil in oxygen-18; and
measuring a third group of three calibrated standard water samples before and after measuring one or more third vials of urine samples, wherein an isotopic composition of the second group of standard water samples spans less than 191.0 per mil in deuterium and less than 26.3 per mil in oxygen-18.

16. The method of claim 11, wherein:
the amount of the deuterium and the amount of the oxygen-18 in the DLW dose are each less than a typical DLW dose; and
there is no decrease in an accuracy of the determined one or more metabolic parameters of the user when the DLW dose is used instead of the typical DLW dose.

17. The method of claim 11, wherein:
there is no correlation between the DLW dose and an accuracy of the one or more metabolic parameters.

18. The method of claim 1, wherein:
test results for a graph showing experimental results of total energy expenditure standard deviation in kilocalories per a plurality of dosages of oxygen-18 less than the DLW dose of 0.18 grams per kilogram of the body water of the user show an R-squared value of about 0.0232.

19. The method of claim 1, wherein:
test results for a graph showing experimental results of total energy expenditure standard deviation in kilocalories per a plurality of dosages of deuterium less than the DLW dose of 0.12 grams per kilogram of the body water of the user show an R-squared value of about 0.0285.

20. The method of claim 11, further comprising:
receiving body weight information of each of the plurality of users; and
providing, to each user, a customized amount of deuterium and oxygen-18 in the DLW dose based on the body weight information.
